(19) United States
(12) Patent Application Publication
(10) Pub. No.: US 2012/0065649 A1
(43) Pub. Date: Mar. 15, 2012

(54) Surgical Mesh

(76) Inventor: Jeffrey C. Towler, Wilmington, DE (US)

(21) Appl. No.: 13/228,775

(22) Filed: Sep. 9, 2011

Related U.S. Application Data
(60) Provisional application No. 61/381,293, filed on Sep. 9, 2010.

(51) Int. Cl. A61B 17/03

(52) U.S. Cl. 606/151

(57) ABSTRACT

An extremely thin surgical mesh with the requisite strength for soft tissue repair deliverable to a surgical site through minimally invasive techniques is provided. Pre-packaged forms of the surgical mesh as well as methods of production and use are also provided.

### CROSS REFERENCE TO RELATED APPLICATIONS

This application claims priority to provisional application U.S. Ser. No. 61/381,293, filed Sep. 9, 2010.

### BACKGROUND OF THE INVENTION

Surgical mesh is used routinely in the repair and restoration of living tissue. Surgical mesh is used to support and/or reinforce damaged or weakened tissues of the body. Surgical mesh is used often, for example, in hernia repair operations.

Various surgical meshes placed laparoscopically or via open surgical techniques are described in U.S. Pat. Nos. 2,671,444; 3,054,406; and 4,452,245.

U.S. Pat. Nos. 6,042,592; 6,375,662; and 6,669,706 disclose a thin woven mesh fabric of resin encapsulated multifilament yarns with a thickness range of about 0.05 millimeters to about 0.50 millimeters suggested to be useful in minimally invasive surgical procedures for repairing and/or reinforcing tissue such as during hernia repair. Introduction and delivery of this surgical mesh into the body using such devices as trocars, cannulas and needle delivery systems is disclosed.

Published U.S. Patent Application No. 2009/0125041 discloses a pre-rolled surgical mesh adapted for insertion into the abdominal cavity double and rolled from two opposite directions, one toward the other.

### SUMMARY OF THE INVENTION

An aspect of the present invention relates to a surgical mesh comprising at least one nonwoven layer designed to be extremely thin for delivery to the patient via a thin delivery device while retaining the requisite strength for soft tissue repair. The surgical mesh can be provided having a very small cross-sectional area by having a rolled or folded or similar configuration Another aspect a of this surgical mesh for use in surgical repair of damaged or weakened tissue is the attachment of sutures that are threaded through the mesh and also delivered via the thin delivery device.

Another aspect of the present invention relates to an article of manufacture comprising this extremely thin surgical mesh rolled, folded, or otherwise configured to be pre-packaged in a containment housing for easier, time saving use by the surgeon in the operating room. Sutures may be attached to the surgical mesh or integral to it.

The present invention also relates to a means to increase the force necessary to pull or tear or otherwise remove an attachment means from the surgical mesh to which it is affixed. The inclusion of macroscopic apertures or load distribution means increases suture retention and similar load bearing characteristics. Thus, the method of increasing the load carrying capability of the mesh is also provided herein.

Another aspect of the present invention relates to a method for repairing damaged or weakened tissues of the body comprising delivering the rolled, folded, or otherwise configured, extremely thin surgical mesh to a surgical site via a thin delivery device; deploying the surgical mesh at the surgical site; and suturing the surgical mesh to the damaged or weakened tissue.

### BRIEF DESCRIPTION OF THE FIGURES

In the figures in which like reference designations indicate like elements.

FIG. 1 is a schematic of a top view of a surgical mesh.

FIG. 2 is a top view of a surgical mesh having multiple fixation means and multiple load distribution means.

FIG. 3 is a top view of a surgical mesh having multiple integral fixation means and multiple load distribution means.

FIG. 4A is a top view of an elliptical surgical mesh having multiple fixation means and multiple load distribution means.

FIG. 4B is a top view of a surgical mesh that has been folded along a longitudinal axis.

FIG. 4C is a top view of a folded and then rolled surgical mesh positioned for insertion into a containment housing.

FIG. 5 is a schematic depicting how the radius of contact was determined in the mesh tension test method.

FIG. 6 is a cross-sectional SEM of a multi-layer mesh article having a tight microstructure layer and a more open microstructure layer.

FIG. 7 is a graph of mesh orientation angle and suture pull-out force as a function of elliptical aperture aspect ratio.

FIG. 8 is a graph of tensile test displacement versus suture pull-out as a function of slit width.

FIG. 9 is a graph of tensile test displacement versus suture pull-out as a function of “hat” shaped slit width.

### DETAILED DESCRIPTION OF THE INVENTION

The present invention provides a surgical mesh that is designed to be particularly useful in minimally invasive surgical procedures for repairing and/or reinforcing tissue, such as, but not limited to, hernia repair. The thin, strong surgical meshes of the present invention may also be useful for minimally invasive laparoscopic technique to correct vaginal prolapse, stress urinary incontinence, or similar pelvic floor disorder. Moreover, the present invention may be applicable to other emerging minimally invasive techniques to treat hernia or similar soft tissue defects such as Single Incision Laparoscopy (SILS), Natural Orifice Translumenal Endoscopy (NOTES).

The thin profile of the mesh of the present invention allows for rolling or folding or other configurations of the surgical mesh having small cross-sectional areas for delivery to the body via a thin delivery device such as a trocar, cannula, needle or the like. By thin profile, it is meant a mesh having a thickness of 0.013 cm or less. By thin delivery device, it is meant a delivery device with an outside diameter of 12 mm or less. Some desirable thin delivery devices have an outside diameter of 6 mm or less.

The surgical mesh of the present invention comprises at least one nonwoven layer that reduces adhesion to tissue. By nonwoven it is meant a layer with a sheet or web structure held together by entangled or interconnected strands or fibers or fibrils. Entanglement or interconnection may be produced mechanically, thermally, or chemically, or may exist inherently within the material of the nonwoven layer. The nonwoven mesh layer comprises a flat, porous sheet made directly from separate fibers (such as polyester, Teflon®, polyurethane, polyacrylonitrile, or cellulose) or from molten plastic, or plastic film (such as but not limited to polyurethane, Teflon®, polytetrafluoroethylene (PTFE), or polymethyl methacrylate). The nonwoven layer of surgical mesh is not made by weaving or knitting and does not require converting the fibers to yarn. In general, nonwoven fabrics were believed to exhibit insufficient strength for purposes of a surgical mesh. However, the nonwoven mesh described herein is both very thin and strong. By strong it is meant that it has a strength of at least 16 newtons/cm. By thin it is meant that it has a thickness of less that 0.01 cm. Also the mesh described herein resists adhesion by inhibiting visceral attachment when used in procedures in which the mesh is implanted.

The surgical mesh of the present discovery comprises expanded PTFE (ePTFE) which may be produced via processes known to one skilled in the art and based on U.S. Pat. No. 3,953,566. The specific properties of the ePTFE films used herein were tailored by the choice of PTFE resin and process conditions. To restrict tissue ingrowth, the pore size of the resulting ePTFE film should be less than the size of the cells to which it will be exposed. Typically, this requires the mesh to have an average pore size of 13 μm or less.

Conventional surgical meshes have an open layer, such as a knitted or woven fiber construct, that provides the requisite strength attached to which an ePTFE or resorbable layer thereby creates the visceral side. In contrast, the present discovery is a very thin, visceral side barrier layer that is also capable of being the load bearing layer. This visceral side is useful in lap ventral hernia repair. The barrier layers on most conventional surgical patches is thin but very weak; typically having an average matrix tensile strength of less than 5 kpsi. One embodiment of the present discovery is as a thin single layer construct that has an average matrix tensile strength of about 40 kpsi or more. Some embodiments may have an average matrix tensile of 50 kpsi ore more. Where tissue ingrowth is desirable, such as into the peritoneum, a more open layer may be combined with this thin barrier layer. Where tissue ingrowth is undesirable, such as to the underlying viscera in an intraperitoneal hernia repair, the barrier layer surface should have an average pore size of approximately less than 13 μm. Also desirable are barrier surfaces having an average pore size of approximately 7 μm or less. Also desirable are barrier surfaces having an average pore size of approximately 4 μm or less. Where tissue ingrowth is desirable, the more open surface of the mesh should have an average pore size of approximately of 13 μm or greater.

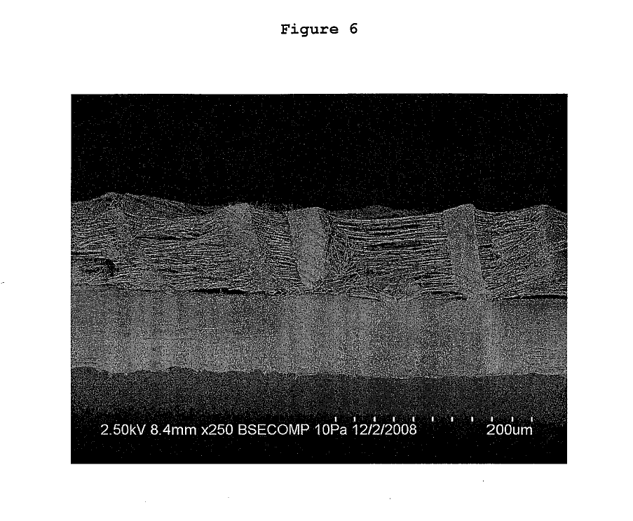

In some embodiments described herein, the microporous membrane structures may be asymmetric. By “asymmetric” it is meant that the microporous membrane structure comprises multiple regions through the thickness of the structure, and at least one region has a microstructure that is different from the microstructure of a second region. In one embodiment, an asymmetric porous membrane comprises multiple regions through the thickness of the structure in the form of layers, such as the layers of an expanded fluoropolymer. For example, a multilayer, expanded polytetrafluoroethylene (ePTFE) membrane may comprise two regions through the thickness of the structure having different microstructures where at least two of the membrane layers have a different microstructure as shown in FIG. 6. In some embodiments, the asymmetric membrane may have three or more membrane regions, or a gradient of microstructure from one interface or surface to another.

As exemplified in the schematic illustration of FIG. 6, the microporous membrane comprises a multi-layer construct having a first microporous membrane region (60) and a second microporous membrane region (65) having a microstructure that is different than the first porous membrane region. In some embodiments, the second porous membrane region (65) may have a more open structure than the first porous membrane region (60). Optionally, additional microporous membrane regions may be included to meet desired mesh requirements.

In some instances, the mesh may have macroscopic pores or open apertures that may be uniformly or non-uniformly distributed across its surface. In the case of lap ventral hernia repair, such open apertures are believed to enhance ingrowth. A desirable percent open area minimum should be about 5% open area. A desirable percent open are maximum should be about 40% open area. In other applications, such as for non-intraperitoneal applications, the maximum percent open area may be as high as 95% open area or even more. These open apertures may take a variety of shapes and may vary based on the particular requirements of each application of the present invention.

The surgical mesh may comprise additional materials. When the mesh is a microporous fluoropolymer or microporous biocompatible polymer, a second material may be imbibed into microstructure to impart additional functionality. For instance, a hydrogel may be imbibed into a microporous ePTFE mesh to enhance cell ingrowth. Optionally, a second material may be coated onto the external surface of the microporous mesh or applied to the internal surfaces of the microstructure of the microporous mesh. Coating materials such as, but not limited to, antibiotic or antiseptic materials may be useful to resist infection. The coating material, rheology, and process parameters can be adjusted to control the amount of material that is deposited on the available internal and/or external mesh surfaces. A broad range of complementary materials may be carried by or included in the present mesh invention to meet the needs of numerous end applications.

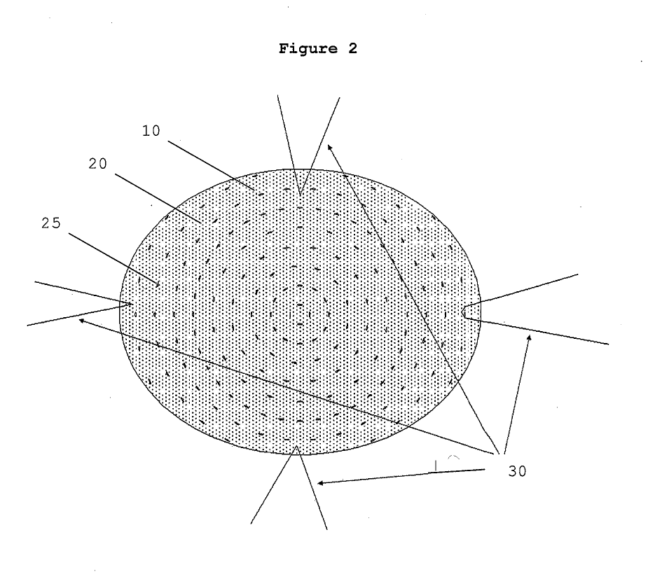

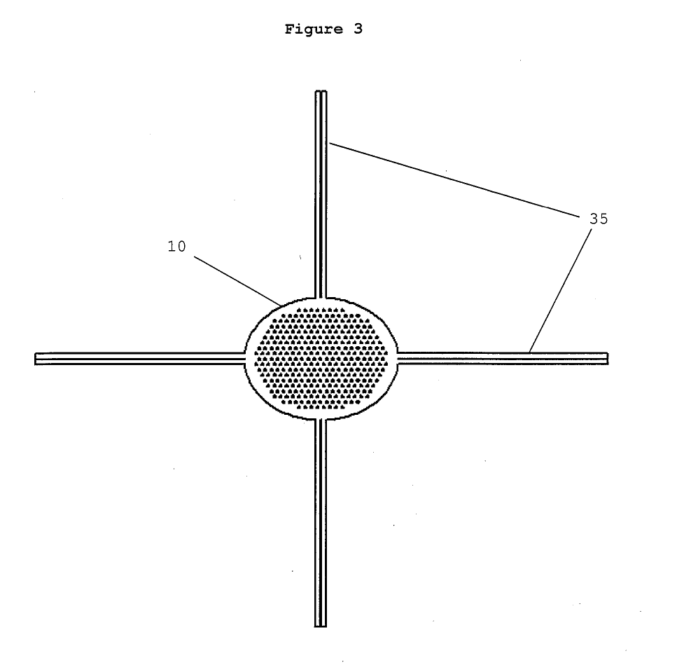

Sutures may be provided with the mesh of the present invention to facilitate surgical placement and securement. FIG. 2 shows a mesh having pre-installed sutures (30) that act as attachment means. Additional attachment means can be employed, such as the inclusion of integral sutures (35) as shown in FIG. 3. The attachment means must be able to initially secure the mesh patch while additional attachments can be made by the surgeon. Possible attachment means include, but are not limited to, staples, tacks, sutures, and adhesives. In hernia repair applications, sutures provide the initial anchoring while tacks are commonly employed to ensure the mesh is sufficiently ‘laying flat’ to the peritoneum.

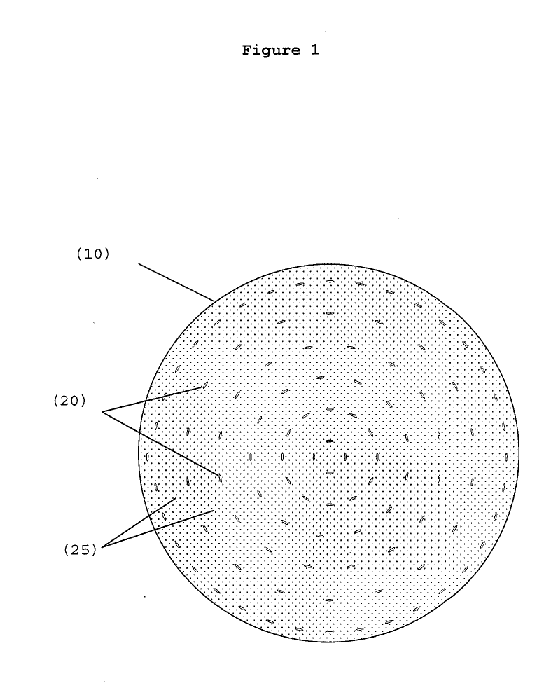

The surgical mesh further comprises a means for load distribution within the surgical mesh to increase retention force when placed by the surgeon. The load distribution means of the present invention is effective with a range if attachment means including those described above. The load distribution means of the present invention are macroscopic apertures hereafter called “means” in the mesh, such as but not limited to slits, holes, ellipses, and other cut-outs. FIG. 1 shows a circular surgical mesh (10) having both large load distribution means (20) and small load distribution means (25). In one embodiment, the load distribution means comprise a plurality of small cuts or slits placed in the surgical mesh. When an attachment means (30), such as a suture, penetrates the mesh and a suture load is applied, the load distribution means effectively increase the force required for suture pullout or tear through the mesh. When slits are used as the load distribution means, the preferred orientation of the slit is perpendicular to the direction to which the suture load is applied. When the load distribution means are preformed ellipses in the mesh, the preferred orientation of the longitudinal axis of the ellipse is perpendicular to the direction to which the suture load is applied. Moreover, by threading the sutures through these premade cuts or ellipses, or other shapes, the tearing and/or ripping of the surgical mesh is inhibited or prevented.

In one embodiment, the surgical mesh further comprises a bioabsorbable portion or ring for stiffening. One skilled in the art will realize that many bioabsorbable materials may be used such, but not limited to, that described in U.S. Pat. No. 6,165,217 The Bioabsorbable portion may be on the edge of the mesh patch or may be at any desirable position, such as it right on the edge or in a certain distance within the edge. In some instances, the bioabsorbable portion may be located at least partially around the periphery of the patch to facilitate deployment once in position within the body. Other deployment aides may also be used such as, but not limited to, wires, ribs, and other stiffening agents.

Depending on the surgical application, the mesh may have an area greater than 100 cm2 and yet still need to be delivered laparoscopically. A typical mesh patch for ventral hernia repair may be an ellipsoidal shape approximately 19 cm long by approximately 15 cm wide. To facilitate delivery, the thin, strong, mesh patch of the present invention may be rolled along its long axis in order to create a small cross-section for insertion into the laparoscopic delivery tool. The present invention allows a large patch such as this to be delivered via a trocar port having, nominally, a 5 mm inside diameter. Smaller size mesh patches can be delivered by an even smaller trocar. In other embodiments where a larger patch is required, a larger inside diameter trocar port may be needed. In alternate embodiments, patch thickness may be varied in order to accommodate larger size patches in smaller trocars.

The present invention also provides an article of manufacture comprising the extremely thin surgical mesh pre-packaged in a containment housing. Pre-packaging of the surgical mesh of the present invention into a containment housing provides for easier, immediate use by the surgeon in the operating room. In one embodiment, the containment housing has an external diameter of less than 5 mm. In the article of manufacture at least a portion of the thin surgical mesh is confined within the containment housing.

To package the mesh within a containment housing, the mesh may be tightly rolled around a small mandrel and the mandrel then removed. Alternatively, the mesh may be rolled without the aid of a mandrel, folded, or otherwise compacted provided that the end result is a mesh conformation that located within a containment housing. Suitable containment housings may be hollow polymeric tubes (e.g. a drinking straw), a thin wrapped film (e.g. a polymeric film), wrapped threads (e.g. a coil-like wrap of a thin fiber or thread), wrapped thin films, and/or any other suitable package that holds the tightly rolled or folded or compacted mesh so that it can be subsequently slid or moved into a device. Containment housings may be made from a range of materials including polymers, biocompatible polymers, bioabsorbable polymers, metal, organic materials, and the like.

The present invention also provides a method for repairing damaged or weakened tissues of the body by delivering the pre-rolled or prepacked surgical mesh of the present invention to a surgical site via a thin delivery device. Examples of thin delivery devices include, but are not limited to cannulas, trocars and needles. In one embodiment the thin delivery device has a diameter of 10 mm or less.

Repairing damaged or weakened tissues requires a relatively strong mesh. For example with a ventral hernia repair, the present invention can provide a 15 cm by 19 cm elliptical mesh having a mesh tension greater than 32 N/cm and yet be thin enough to be rolled up for delivery through a 5 mm trocar port. In the case of this 32 N/cm mesh, the thickness was about 0.01 cm. When an adhesion barrier is desired, a thinner mesh may be employed having a mesh tension greater than 16 N/cm. In which case, an even larger mesh will fit within the same 5 mm delivery trocar port. Or a similar size (elliptical shape measuring 15 cm×19 cm) could be packaged into a trocar having a diameter less than 5 mm. A 4 mm OD trocar may be used. Or a 3 mm OD trocar may be used.

The packaged mesh may be moved into the surgical device (e.g. trocar, cannula, or needle) by aligning the end of the containment housing with the open end of the surgical device and pushing, with a suitable tool, the pre-rolled mesh from the containment housing into the surgical device. Once inside the surgical tool, the containment housing may be removed. An alternate approach is to design the containment housing to fit inside the surgical device, in which case, the containment housing only needs to be slid into the surgical device in its entirety. Then the mesh can be pushed or pulled from the surgical instrucment after the surigical instrument is placed within the patient's body.

The packaged mesh may be sterilized while in the containment housing, or prior to insertion into the containment housing, or after relocation to the surgical device. Any suitable sterilization means may be used, including but not limited to γ-radiation, steam, ethylene oxide (EtO), and peroxide.

In one embodiment, the surgical mesh of the present invention is used in hernia repair. In this embodiment, the surgical mesh is delivered into the preperitoneal cavity of a patient via a thin delivery device. A second small needle cannula can be inserted into the preperitoneal cavity to insufflate the area with carbon dioxide. The hernia sac is dissected free and ligated. A laparoscope is also inserted via a cannula for visualization during the procedure. The surgical mesh, upon being released from the delivery device can be unfurled over the transversalis facia and then manipulated to cover the myopectineal cavity. The surgical mesh is then sutured or stapled over the herniated region to provide added support to the tissue of the preperitoneal cavity. The delivery device is removed and the access location closed. Over time, the surgical mesh is assimilated by the body tissue.

In another embodiment, the surgical mesh of the present invention may be used in any other laparoscopic procedure where a repair patch is needs to be delivered via a minimally invasive surgical means.

In other surgical procedures, a different size or diameter delivery device may be warranted. The design parameters of the present invention may be changed accordingly. If the sole purpose is as an adhesion barrier, then a strength less than 16 N/cm may be useful in which case either larger patches may be deployed from the same size delivery device, or a smaller delivery device could be used, or both.

### Test Methods

### Mesh Tension

Mesh tensions for the examples described below were measured in accordance with ASTM D3787 based on the measured force and the radius of contact (r_{contact}) with the ball.

Mesh tension=Force/2*π*r_{contact}

The radius of contact (r_{contact}) was determined using contact paper as follows:

A nip impression kit (10002002 Nip Impression Kit from Metso Paper, P.O. Box 155, Ivy Industrial Park, Clarks Summit, Pa. 18411)) is used to measure the length of ball contact with the mesh. This kit contains a roll of carbon paper and a roll of plain white paper, which can be dispensed so that any given length of both will be obtained with the carbon side flush against the white paper. The two papers are inserted io between the ball and the mesh. As the load or pressure is applied between the ball and the mesh the carbon paper will leave an ink mark impression in the shape of the knit on the white paper. The impression length on the white paper is measured with a steel ruler with 0.5 mm increments.

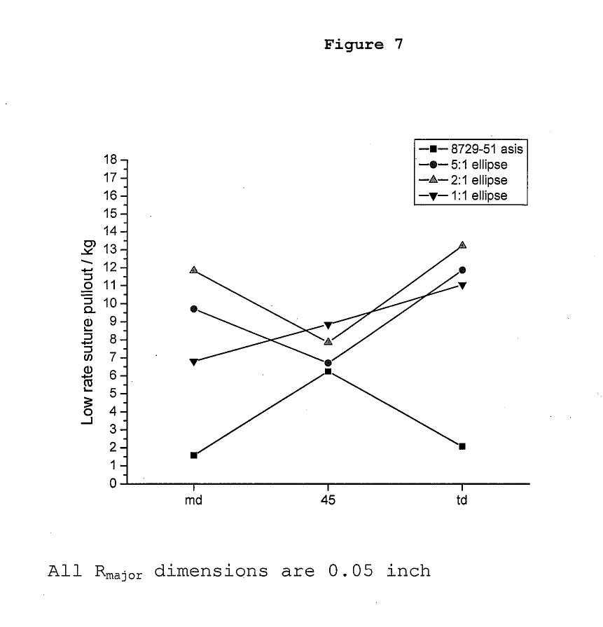

The length of ball contact and the radius of the ball are used to determine the angle of contact as shown in FIG. 7.

2γ=length of ball contact/r_{ball}

γ=(length of ball contact/r_{ball})/2

r_{contact}=r_{ball}*sin(γ)
where, 2γ=angle of contact
   r_{ball}=radius of the ball
   r_{contact}=radius of contact

### Suture Retention

Suture retention is a mechanical property reflecting the articles mechanical resistance under tension at a suture site placed in the article. To represent the load applied by a suture at a suture site, a small pin fixture was used in which a pin (typically 0.020″, or multiple pins) was pressed through a 1 inch wide strip of the test article. The coupon/attached-pin-fixture combination is attached in a tensile test apparatus such as an Instron Tensile Tester. The crosshead speed was set to 200 mm/min. For purposes of this measure, the maximum force exhibited was as the ‘suture retention’ strength. However, other parameters shown in the stress-strain graphs in FIGS. 6 and 7 may also be used to define the reinforcement phenomenon described herein.

The following nonlimiting examples are provided to further illustrate the present invention.

### Matrix Tensile Strength

Tensile testing was carried out on a tensile test machine operating under displacement control at constant speed. The thickness of each cut sample was determined to be the mean of three measurements made using a snap gauge at three different locations within the length of the sample.

Because the inventive and control samples are porous materials, tensile strength values were converted to matrix tensile strength values in order to compensate for differing degrees of porosity. Matrix tensile strength was obtained by multiplying the tensile strength of each individual sample, determined as described above, by the ratio of the 2.2 g/cm³ density of solid, non-porous PTFE to the density of the porous sample.

### EXAMPLES

### Tape 1

Fine powder of PTFE polymer as described and taught in U.S. Pat. No. 6,541,589, comprising perfluorobutylethylene modifier, was blended with Isopar K (Exxon Mobil Corp., Fairfax, Va.) in the proportion of 0.200 g/g of fine powder. The lubricated powder was compressed in a cylinder to form a pellet and placed into an oven set at 70° C. for approximately 8 hours. The compressed and heated pellet was ram extruded to produce an extrudate tape approximately 15.2 cm wide by 0.75 mm thick. The tape was then calendered between compression rolls, distended, and dried to yield a tape having matrix tensile strengths of 6 kpsi (machine direction)×6 kpsi (transverse direction). The side of the resultant asymmetric mesh surface corresponding to Tape1 is herein considered the tight-structure side.

### Tape 2

Fine powder of PTFE polymer (DuPont, Wilmington, Del.) was blended with Isopar K (Exxon Mobil Corp., Fairfax, Va.) in the proportion of 0.243 g/g of fine powder. The lubricated powder was compressed in a cylinder to form a pellet. The compressed pellet was ram extruded at room temperature to produce an extrudate tape approximately 15.2 cm wide by 0.75 mm thick. The tape was then calendered between compression rolls, set to a temperature of 38° C., to a thickness of 0.28 mm. The tape was then longitudinally distended 8% and dried. The process produced a calendered tape having matrix tensile strengths of 3.2 kpsi (machine direction)×1.4 kpsi (transverse direction). The side of the resultant asymmetric mesh surface corresponding to Tape2 is herein considered the open-structure side.

### EXAMPLE 1

### Thin Two-Sided Patch

Six layers of Tape1 were stacked on top of one another, each layer being 90 degrees offset from the previous. The stack was io compressed and laminated together under high vacuum (<29″ Hg) at 309° C. and 100 k-lbs force for 4 minutes to full density on OEM press Model VAC-Q-LAM-1/75/14X13/2/4.0″/E370C/N/N/N-C-480V (OEM Press Systems Inc., 311 S. Highland Ave., Fullerton, Calif. 92832). The compressed stack was allowed to cool and then cut into an 8.5 inch diameter circle.

The circular sample was gripped around the periphery and radially expanded at 300° C. and an axial expansion rate of 3.0 inch/second to an area expansion of about 11.25:1. The radially expanded sample was then relaxed to achieve a 1.5:1 area reduction. The sample was removed and cut into a 9″×9″ coupon. This process was repeated four times to create four radially expanded PTFE disks.

A mesh was created by combining four radially expanded PTFE disks from above with one layer of Tape2 into a single stacked coupon. The stacked coupon was compressed and laminated together under high vacuum (<29″ Hg) at 309° C. and ˜100 k-lbs force for 4 minutes to approximately full density on OEM press Model VAC-Q-LAM-1/75/14X13/2/4.0″/E370C/N/N/N-C-480V (OEM Press Systems Inc., 311 S. Highland Ave., Fullerton, Calif. 92832). The compressed densified stack was allowed to cool and cut to an 8.5 inch circle. The circular sample was gripped around the periphery and expanded at 300° C. and a rate of 0.2 inch/second axial displacement to an expansion ratio of about 11.25:1. The expanded mesh was then allowed to relax to an area reduction of about 1.5:1. The mesh was then restrained in a convection oven (ESPEC Model SSPH-201, 4141 Central Parkway, Hudsonville, Mich. 49426) at 350° C. for 10 minutes, and then allowed to cool.

A cross-sectional SEM of this microporous expanded asymmetric PTFE mesh article is shown in FIG. 6.

### EXAMPLE 2

### Thin Two-Sided Patch Pre-Sutured with Suture Management

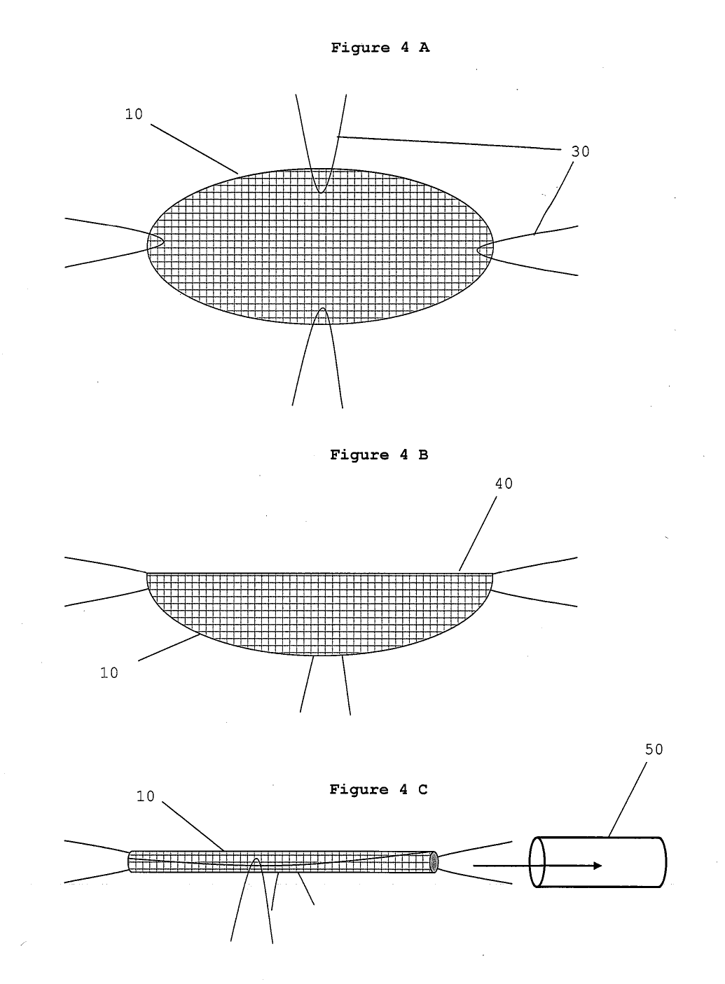

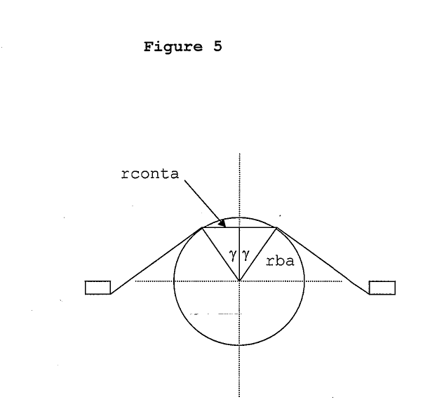

A sample of the mesh from Example 1 was cut into 15 cm×19 cm oval device using CO2 Plotter/Laser (Universal Laser Systems Model PLS6.60-50 16000 M 81^{st} Street, Scottsdale, Ariz. 85260). Then GORE-TEX CV-2 sutures (W.L. Gore and Associates Inc., 301 Airport Road, Elkton, Md. 21921) were looped through at four cardinal locations: 12, 3, 6, and 9 o'clock positions as shown in FIG. 4A. Each suture(30) was passed about 0.5 cm inward from the edge. Each suture was looped io through the device such that the free ends were on the abdominal side of the device. The entry and exit point of each suture loop was about 0.5 cm apart. Next a thin, strong piece of a fluorinated ethylene propylene (FEP)/expanded PTFE (ePTFE) composite film was cut into an approximately 1 cm×0.5 cm rectangle. The expanded PTFE film was prepared in conformance with U.S. Pat. No. 5,476,589A. The FEP layer was approximately 1 mil thick. This cut rectangle was placed on the open side of the sutured mesh so that each exposed suture was covered. These FEP/ePTFE rectangles where then welded to the mesh thereby securing the sutures in place. The welding was accomplished using a soldering gun with a blunt tip and set to 800° F. and hand pressure (Weller WSD161, APEX Tool Group LLC., 14600 York Road Suite A, Sparks; Md. 21152).

Suture Management designed to avoid suture entanglement was accomplished by bundling attached pairs of oriented sutures using coils produced from a “string” of bioabsorable polymer produced in conformance with U.S. Pat. No. 6,165,217. The bioabsorbable film mass was 7 mg/cm2. This film was “cigarette rolled” to produce the “string”. This “string” was then looped around sutures securing the parallel adjacent sutures. Heat (260° F., 10 seconds) was applied via heat gun (Steinel Model HL2010E, 9051 Lyndale Avenue, Bloomington, Minn. 55420) to retract and thermally set the bioabsorbable polymer.

### EXAMPLE 3

### Thin Two-Sided Patch Pre-Sutured Packed in Tube for Delivery Through 5 mm Trocar Port

The sutured mesh article from Example 2 was folded in half across the ellipse minor axis (40) as shown in FIG. 4B. The folded mesh was placed between two small mandrels (or a split mandrel) (New England Precision Grinding, 0.013″×70″ PTFE coated 304SS mandrels, 35 Jeffrey Avenue, Holliston, Mass. 01746-2027) that were chucked on a horizontal rotary drill press and the drill press rotated to roll up sutured mesh device into a tight package around the mandrels. The rolled sutured mesh assembly was removed from the chucks, and the mandrels removed from within the rolled, sutured mesh. The rolled, sutured mesh assembly was inserted into a ˜5.2 mm ID tube (50) (nylon tubing of 0.005″ id.) wall from Grilam as depicted in FIG. 4C. The tube and rolled suture device was inserted into a 5 mm trocar port of ID ˜5.5 mm (Covidien 15 Hampshire Street, Mansfield, Mass. 02048). Deployment of the sutured mesh was demonstrated when the rolled sutured mesh was easily pushed out of the trocar and unrolled onto the table top where is laid relatively flat.

### EXAMPLE 4

### Load Distribution—5:1 Elliptical Aperture

The suture retention effect of creating elliptical apertures was determined using an ePTFE mesh article created in conformance with U.S. Pat. No. 7,306,729. The base ePTFE material had matrix tensile strengths of 48 kpsi and 46 kpsi in the machine and transverse directions, respectively. The material was mounted in a CO2 plotter/laser (Universal Laser Systems Model PLS6.60-50 16000 M 81^{st} Street, Scottsdale, Ariz. 85260). The beam was focused on the plane of the material. In the orientation of the test directions (machine direction, transverse direction, and 45 degree nominally), an ellipse having rₘₐⱼₒᵣ 0.05″ and rₘᵢₙₒᵣ 0.010″ (i.e. 5:1 ratio) was laser cut from the material oriented so the ellipse was substantially parallel to the perimeter of the mesh article. The suture retention measurements were performed by sequentially locating the test pin in a lased aperture in each of the machine, transverse, and 45 degree directions. The results are shown in FIG. 7.

### EXAMPLE 5

### Load Distribution—2:1 Elliptical Aperture

The suture retention effect of creating elliptical apertures was determined using an ePTFE mesh article created in conformance with U.S. Pat. No. 7,306,729. The base ePTFE material had matrix tensile strengths of 48 kpsi and 46 kpsi in the machine and transverse directions, respectively. The material was mounted in a CO2 plotter/laser (Universal Laser Systems Model PLS6.60-50 16000 M 81^{st} Street, Scottsdale, Ariz. 85260). The beam was focused on the plane of the material. In the orientation of the test directions (machine direction, transverse direction, and 45 degree nominally), an ellipse having rₘₐⱼₒᵣ 0.05″ and rₘᵢₙₒᵣ 0.025″ (i.e. 5:1 ratio) was laser cut from the material oriented so the ellipse was substantially parallel to the perimeter of the mesh article. The suture retention measurements were performed by sequentially locating the test pin in a lased aperture in each of the machine, transverse, and 45 degree directions. The results are shown in FIG. 7.

### EXAMPLE 6

### Load Distribution—1:1 Elliptical Aperture

The suture retention effect of creating elliptical apertures was io determined using an ePTFE mesh article created in conformance with U.S. Pat. No. 7,306,729. The base ePTFE material had matrix tensile strengths of 48 kpsi and 46 kpsi in the machine and transverse directions, respectively. The material was mounted in a CO2 plotter/laser (Universal Laser Systems Model PLS6.60-50 16000 M 81^{st} Street, Scottsdale, Ariz. 85260). The beam was focused on the plane of the material. In the orientation of the test directions (machine direction, transverse direction, and 45 degree nominally), an ellipse having rₘₐⱼₒᵣ 0.05″ and rₘᵢₙₒᵣ 0.050″ (i.e. 5:1 ratio) was laser cut from the material oriented so the ellipse was substantially parallel to the perimeter of the mesh article. The suture retention measurements were performed by sequentially locating the test pin in a lased aperture in each of the machine, transverse, and 45 degree directions. The results are shown in FIG. 7.

### EXAMPLE 7

### Load Distribution—Control, No Elliptical Aperture

The suture retention effect of creating elliptical apertures was determined using an ePTFE mesh article created in conformance with U.S. Pat. No. 7,306,729. The base ePTFE material had matrix tensile strengths of 48 kpsi and 46 kpsi in the machine and transverse directions, respectively. This control sample was tested by pressing the test pin through the mesh article in locations corresponding to each of the machine, transverse, and 45 degree directions. The results are shown in FIG. 7.

### EXAMPLE 8

### Load Distribution—Slit Element

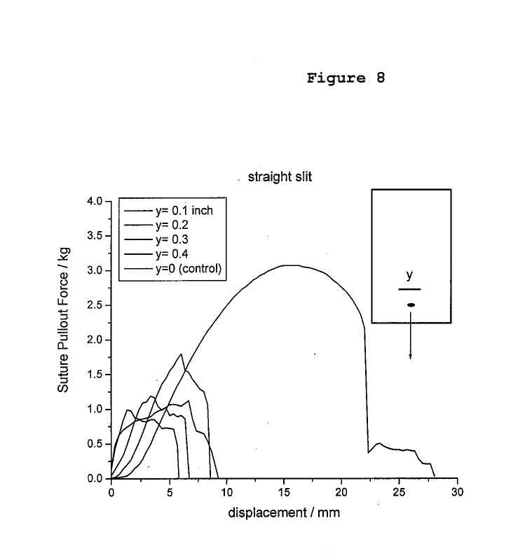

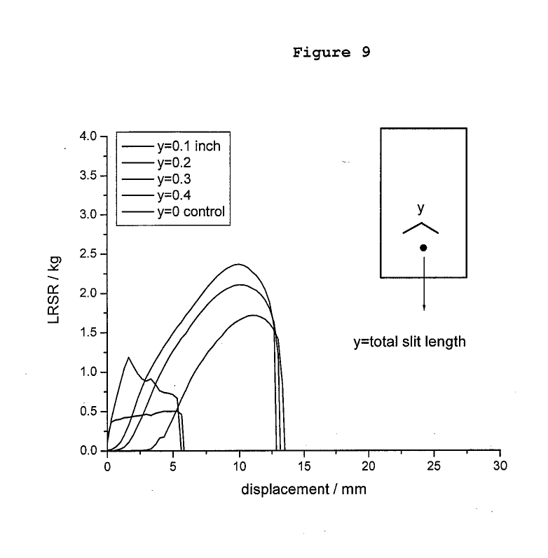

The effect on suture retention of creating a small slit near the suture location determined using an ePTFE mesh article created in conformance with U.S. Pat. No. 7,306,729. The base ePTFE material had matrix tensile strengths of 48 kpsi and 46 kpsi in the machine and transverse directions, respectively. A small slit cut was cut with a razor blade approximately 0.5 cm in from and parallel to the edge of the mesh article. The test pin was then pressed through the mesh article at a location between the slit and the edge of the article. The tensile properties were measured. FIG. 8 shows the suture pull-out tensile results as a function of slit length compared to a control sample having no slit.

### EXAMPLE 9

### Load Distribution—“Hat” Element

The effect on suture retention of creating a small “hat” shaped slit near the suture location determined using an ePTFE mesh article created in conformance with U.S. Pat. No. 7,306,729. The base ePTFE material had matrix tensile strengths of 48 kpsi and 46 kpsi in the machine and transverse directions, respectively. A small “hat” shaped slit cut was cut with a razor blade approximately 0.5 cm in from and parallel to the edge of the mesh article. The test pin was then pressed through the mesh article at a location between the “hat” shaped slit and the edge of the article. The tensile properties were measured. FIG. 8 shows the suture pull-out tensile results as a function of the “hat” shaped slit length compared to a control sample having no slit.

What is claimed is:

1. A surgical mesh comprising at least one nonwoven layer which resists adhesion to tissue while retaining the requisite strength for soft tissue repair wherein the mesh comprises a profile sufficiently thin to be delivered via a thin delivery device.

2. A surgical mesh comprising at least one nonwoven layer that resists adhesion to tissue while retaining the requisite strength for soft tissue repair, said mesh being sufficiently thin to be delivered via a thin delivery device and pre-packaged for delivery.

3. The surgical mesh of claim 2 wherein the surgical mesh is pre-rolled.

4. The surgical mesh of claim 2 wherein the surgical mesh is pre-folded.

5. A medical device comprising:
a. A surgical mesh formed into a rolled configuration;
b. a housing which packages the rolled mesh for delivery.

6. The medical device of claim 5 wherein the housing has an outer diameter of less than 6 mm.

7. The device of claim 6 further comprising pre-attached sutures.

8. The device of claim 1 further comprising oriented apertures for enhanced fixation.

9. The device of claim 1 wherein the surgical mesh has a thickness of about 0.013 cm or less.

10. The device of claim 2 wherein the surgical mesh has a thickness of about 0.013 cm or less.

11. The device of claim 1 wherein the surgical mesh comprises at least one non-woven layer of expanded PTFE.

12. The device of claim 2 wherein the surgical mesh comprises at least one non-woven layer of expanded PTFE.
